(19) United States
(12) Patent Application Publication
(10) Pub. No.: US 2013/0184611 A1
(43) Pub. Date: Jul. 18, 2013

(54) SYSTEM AND APPARATUS FOR POSTURE AND BODY POSITION CORRECTION AND IMPROVEMENT THROUGH A COMPUTER-ASSISTED BIOFEEDBACK SYSTEM

(71) Applicant: Andrew Nichols, Coconut Grove, FL (US)

(72) Inventor: Andrew Nichols, Coconut Grove, FL (US)

(21) Appl. No.: 13/549,057

(22) Filed: Jul. 13, 2012

Related U.S. Application Data
(60) Provisional application No. 61/507,255, filed on Jul. 13, 2011.

(51) Int. Cl. A61B 5/04

(52) U.S. Cl. CPC A61B 5/04
USPC 600/587

(57) ABSTRACT

A wireless, programmable biofeedback system, including one or more sensor/transceiver biofeedback devices, a controller/ transceiver biofeedback device, and a wireless communication link that operationally connects the sensor/transceiver biofeedback device to the controller/transceiver biofeedback device(s). A gyroscopic mechanism may be operationally connected to the sensor/transceiver biofeedback device, as may a distance measuring mechanism, memory, and/or a microprocessor. The sensor/transceiver biofeedback devices attached to a user's body to detect changes in position of the part of the user's body to which the device is attached, and communicate such changes wirelessly to the controller/transceiver biofeedback device. One or more indicating means provide stimulus to train and condition users to maintain or achieve certain postures and/or other body positions that the user can set, monitor and modify.

### CROSS-REFERENCE TO RELATED APPLICATIONS

This patent application claims priority to co-pending U.S. Provisional Patent Application Ser. No. 61/507,255, filed on Jul. 13, 2011.

### TECHNICAL FIELD

The novel technology relates generally to the field of personal health and self-improvement, and, more specifically, to a biofeedback system, which may include a related software system, for training people to achieve better posture and other body position goals.

### BACKGROUND

Maintaining good posture and avoiding slouching and stooping is increasingly important to all age groups—especially with aging populations and people of all ages spending more and more time in front of computers and televisions, or in other sedentary pursuits. Improving stance, posture and body position can have significant benefits for health, safety and performance. Posture-related problems inevitably impact on both appearance and health. Poor posture is also the most common source of pain and poor performance in activities. It can result in changes in muscle length, muscle imbalances, faulty movements, and ultimately, pain; creating problems that may not surface for years. Improving posture is easier when a person is younger with a more pliable. Unfortunately, most people wait until they have serious functional problems before they start addressing posture-related issues. Good posture promotes more energy by allowing a person to breathe easier, and do more with less effort as muscles work better when well aligned.

Occupational musculoskeletal injuries represent a pandemic problem worldwide with an enormous, yearly effect on the quality of millions of lives. In the workplace, poor back posture is a major cause of pain, workplace stress and repetitive strain injury resulting in lost time, reduced employee productivity, poor employee health, low morale, and ultimately higher costs. In the U.S. alone, non-accidental, workplace injuries (much of it posture related) costs the economy tens of billions of dollars annually. Poor body mechanics (such as slouching in an office chair), prolonged activity, repetitive motions, and fatigue are major contributors to these injuries. By way of example, in 2006 there was an estimated 357,160 cases of work-related musculoskeletal disorders resulted in lost workdays according to the U.S. Bureau of Labor Statistics. For the same period, the yearly cost associated with common pain conditions and lost productivity was estimated at $61.2 billion.

As a subset of posture-related injuries, with so many people almost permanently attached to computers, there is an affliction of forward-head posture problems. It is estimated that approximately 90% of people in the U.S. have some degree of forward head posture; and for every inch the head moves forward, it gains approximately 10 pounds in weight in terms of strain on the upper back and neck, which have to work incrementally harder to keep the chin from dropping onto a person's chest. Aside from the enormous strain on the body, forward-head posture reduces vital lung capacity by as much as 30% and also adversely affects the gastrointestinal system, particularly the large intestine.

Recent research published by the British Chiropractic Association describes the U.K. population's sedentary lifestyle spiral as creating a nation of slouch potatoes, taking a massive health toll. Over 56% of British people surveyed believe they have bad posture, with more than 60% of 16-18-year-olds found to have undesirable posture, and more than 72% of respondents admitted that they had suffered from back pain at some point. Back pain, particularly common among people between the ages of 35 and 55, is a major cause of workplace absence. Researchers have also found a direct link between poor posture and blood pressure issues based on a neural connection between neck muscles and a part of the brainstem that plays a crucial role in regulating heart rate and blood pressure.

Both in and out of the workplace, with a aging population, the evidence is visually clear of gravity's effects. Along with slouching, as people age, they also start growing down (i.e., shrinking). Unless a person takes steps to reverse this trend, by the time he or she reaches the 70s, there can be a 2-3″ loss in height. Much of that loss is directly correlated with age-related posture deterioration, which begins to set in as early as age 25. At that point, the soft tissues begin to weaken and give in to the relentless pull of gravity. For most people, the first visible signs of poor posture back support is a forward head and rounded shoulders. If a person does not begin corrective posture exercises and take other steps to correct posture early on, long-terms problems will evolve. Those include rounded shoulders and forward head posture that will gradually develop into a hunched back, a.k.a. hyperkyphosis, which is not only bad from an esthetic point of view (making people look older and smaller), but also sets people up for a host of health problems as they age.

Beyond health problems, good posture also promotes better performance and results in most sports and other physical activities. Learning proper positioning and stances reduces body stress, prevents injuries, and optimizes muscle utilization and output. By example, keeping knees bent and a back straight can help a player hit a better tennis ball; and positioning the hips over the balls of the feet and keeping the legs shoulder-width apart can help a skier better control turns on a downhill course.

Clearly, poor posture has a direct and dramatic impact on the health and well-being of all populations. There are hundreds of billions of dollars of annual direct and indirect costs of poor posture worldwide, including treatment of medical conditions and lost worker productivity. Aside from preventing injuries and health problems, improving posture has a correlative, positive impact on performance in sports and other activities. The present novel technology addresses the compelling need to promote better posture and train users to reach and maintain targeted body positions.

### SUMMARY

The present novel technology relates to biofeedback devices and a system and method for training users to achieve and maintain targeted postures and body positions. While there are posture-improving biofeedback systems on the market, they generally require cumbersome harnesses or other wearable systems that detect changes in tension or distance between two or more physically connected devices.

Miniaturization of gyroscopes, microprocessors, memory chips, and radio frequency identification and other mechanisms have revolutionized the ability to detect, obtain, store, wirelessly communicate, process and display information, including changes in body position. Such detection and communication devices can be affixed to different parts of the human body, and can send information to computing devices, including “smart phones” and PDA's able to process, display and respond to the communicated information.

The current, novel technology uses a sensor/transceiver biofeedback device (Sensor), attached to a user's body, clothing or accessories, to monitor positional change and provide immediate feedback to the user. The Sensor detects changes in body position by an internal gyroscope and in some embodiments by wirelessly measuring distances between two Sensors. The Sensors provide biofeedback training through an internal sound or vibration alarm that is activated when the user deviates from or achieves a targeted position. The targeted positions for Sensors are pre-set by a user through a controller/transceiver biofeedback device (Controller) that is wirelessly linked to each Sensor and may be a programmable cellular phone or other computer device on which the novel software program for the system has been installed. Through the wireless link to each Sensor, the Controller may be programmed to emit a sound and/or vibration alarm at the same time an alarm is activated in the Sensor. Information from the Sensors may be shown on the Controller's internal display screen or an external monitor, and may also be stored in the Controller's memory to illustrate a user's progress over time. One object of the present novel technology is to provide an improved means for training users through the instant alarms and visual displays to attain and maintain targeted posture and/or body position objectives. Related objects and advantages of the present novel technology will be apparent from the following description.

### BRIEF DESCRIPTION OF THE DRAWINGS

FIG. 1 is a schematic diagram of a biofeedback system (1) of a first embodiment of the present novel technology.

FIG. 2A is a Schematic Representation of Certain Internal Components of Devices in the embodiment of FIG. 1.

FIG. 2B is a Schematic Representation of Additional Internal Components of Devices in the embodiment of FIG. 1.

FIG. 3 is a Schematic Representation of Biofeedback System of FIG. 1. FIG. 4A graphically illustrates sample screenshots for portions of the System of FIG. 1.

FIG. 4B graphically illustrates sample screenshots for portions of the System of FIG. 1.

FIG. 5A graphically illustrates sample screenshots for portions of the System of FIG. 1.

FIG. 5B graphically illustrates sample screenshots for portions of the System of FIG. 1.

FIG. 6A graphically illustrates sample screenshots for portions of the System of FIG. 1.

FIG. 6B graphically illustrates sample screenshots for portions of the System of FIG. 1.

FIG. 7A graphically illustrates sample screenshots for portions of the System of FIG. 1.

FIG. 7B graphically illustrates sample screenshots for portions of the System of FIG. 1.

FIG. 8A graphically illustrates sample screenshots for portions of the System of FIG. 1.

FIG. 8B graphically illustrates sample screenshots for portions of the System of FIG. 1.

### DETAILED DESCRIPTION

For the purposes of promoting an understanding of the principles of the novel technology and presenting its currently understood best mode of operation, reference will now be made to the embodiments illustrated in the drawings and specific language will be used to describe the same. It will nevertheless be understood that no limitation of the scope of the novel technology is thereby intended, with such alterations and further modifications in the illustrated devices and such further applications of the principles of the novel technology as illustrated therein being contemplated as would normally occur to one skilled in the art to which the novel technology relates. Some embodiments may omit some of the components of the biofeedback system (1) (5), and some embodiments will include other components as well. The illustrated embodiments in the drawings are intended to be exemplary only.

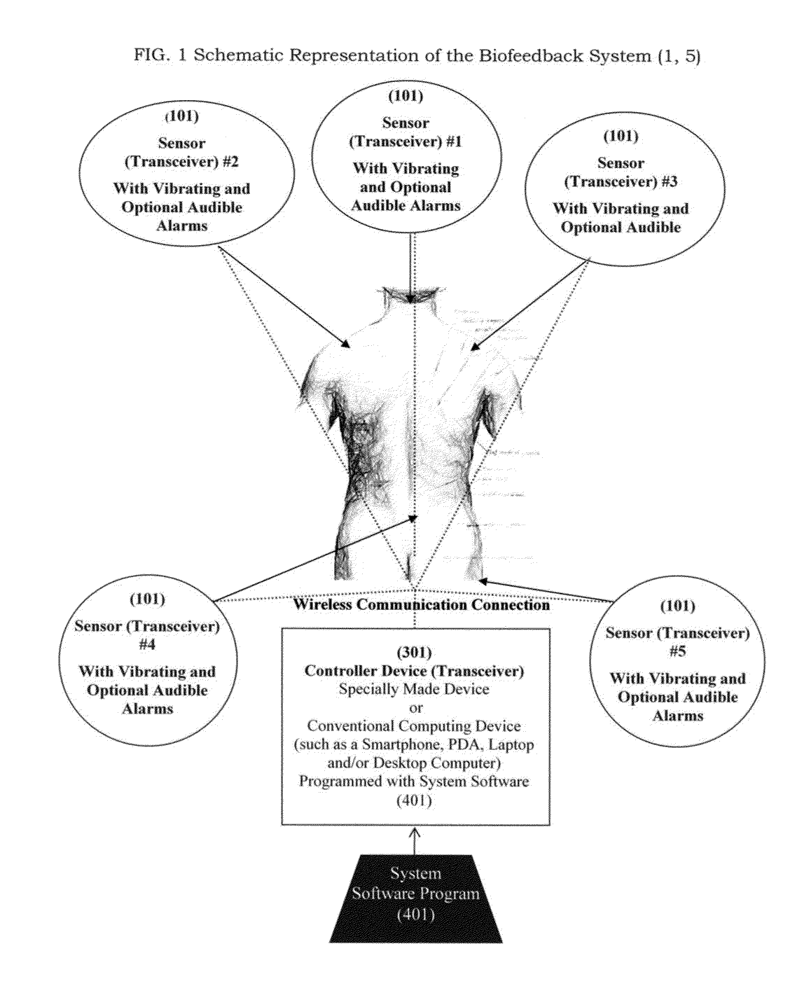

FIG. 1 diagrams the biofeedback system (1) and its component devices and representative parts of the body monitored for positional changes. The biofeedback system (1) includes one or more ‘Sensor’/transceiver biofeedback devices (101) (each a Sensor) programmed to detect changes in posture and body position through an internal gyroscope and/or based on wireless measurement of the distance between another Sensor. Each Sensor has a built-in vibration and/or sound device activated by changes in body position. Such changes are also wirelessly communicated to a remote ‘Controller’/transceiver biofeedback device (301) (Controller) that also has built-in sound and/or vibration devices. The Controller (301) may be a specially-made transceiver device, or a conventional, programmable device on which the biofeedback system software (401) may be installed (e.g. smart-phone, handheld computer, PDA, PC, etc.). The Controller maintains a communication link (201) with each Sensor via Bluetooth, infrared, radio frequency or other technologies. The Sensors (101) are attached to or worn on a person's body (or on clothing or accessories) through adhesion, straps, hair and other clips, bindings and other mechanisms (113). For certain embodiments of the system (5), the system software (Biofeedback System Software) (401) is installed on the Controller (301), which displays on an internal screen or external monitor real-time body position information based on communication from one or more Sensors.

In operation, the system (1) (5) is designed to provide a user with almost immediate feedback about changes in body position and posture. Such biofeedback is used to teach or train a user to avoid undesirable, or achieve desired, body positions and postures. To do so, each Sensor (101), having the form of a fob or like object, is actuated automatically to notify the user by an alarm (vibration and/or sound) once either or both (i) it deviates from and/or achieves, pre-set, horizontal or vertical planes; and/or (ii) it comes within a pre-set range of, or moves more than a pre-set distance from, another Sensor. Once an alarm is actuated in a Sensor, a similar vibration and/or sound alarm may also be wirelessly activated in a Controller (301), and the sound in the Controller may be programmed to be identical to that of the specific Sensor that triggers the initial alarm. By being attached to or worn on specific parts of the body, once an alarm is activated in a Sensor, the user is able to feel the vibration on or about the body part. Each Sensor (101) has an individualized digital signature recognized by the Controller (301) through the wireless communication system (201) so as to distinguish it from other Sensors that may be part of the biofeedback system (1).

The Controller (301) remotely controls the settings for each Sensor (101) through the wireless communication system (201). Settings may include horizontal and/or vertical planes; sound and/or vibration alarms; distances or ranges between Sensors; and associations of each Sensor with a body part. An alarm (audible and/or vibratory) is set off in both the Controller and the Sensor if either of both (x) a body part to which a Sensor is affixed deviates from, or achieves, pre-set horizontal and/or vertical planes; and/or (y) two or more paired Sensors move beyond, or come within, pre-set ranges or distances of each other.

The Sensors (101) are small (e.g., the size of a Quarter or Half Dollar coin), battery powered programmable transceivers. They may be attached to or worn on a user's body or clothes through various means, including adhesive backing, straps, hair and other clips, and bands. Each Sensor (101) may be programmed with a distinct alarm sound and volume. Devices in the system (1, 5) communicate with each other using Bluetooth, WiFi, radio wave, or other technologies (201).

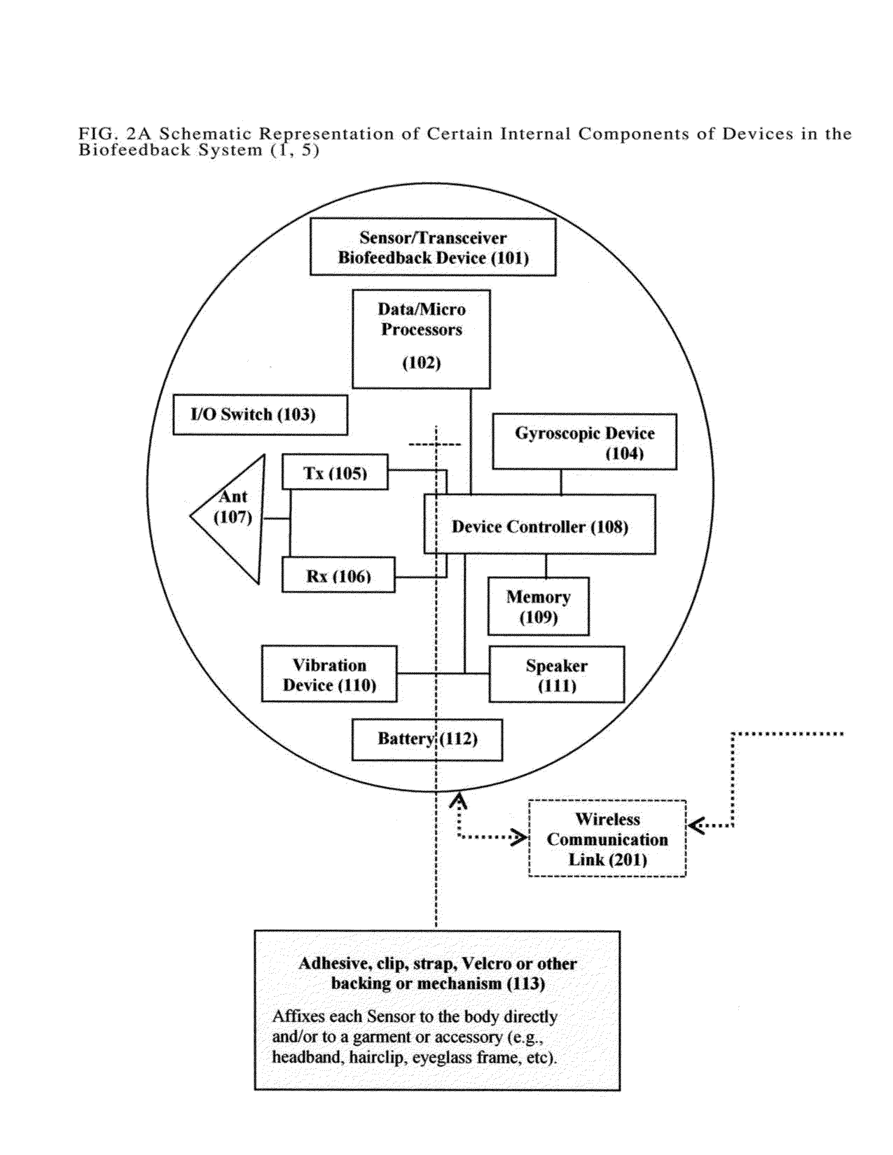

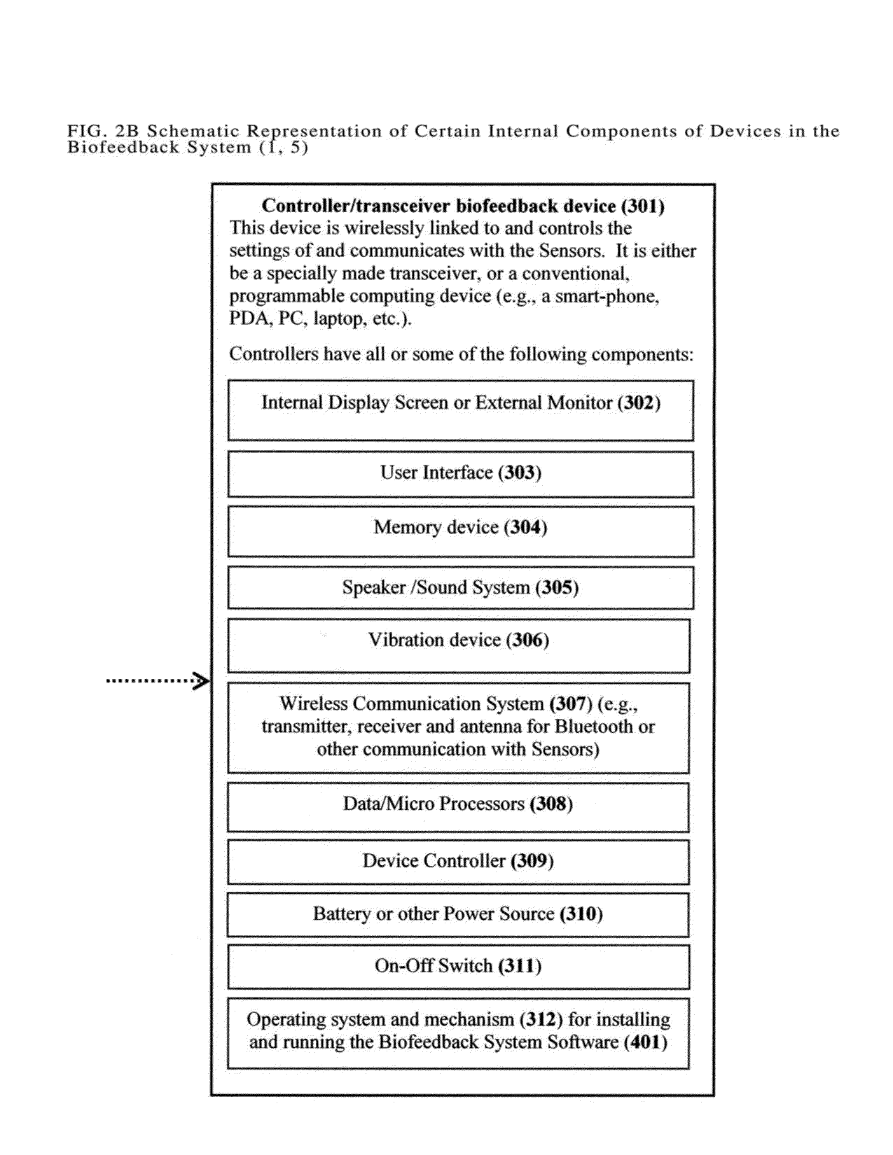

FIG. 2 illustrates certain internal components of the devices included in the personal biofeedback system (1, 5), which are intended to be exemplary only. As shown in FIG. 2, each ‘Sensor’/transceiver biofeedback device (101) includes a housing containing circuitry and other components that may include the following:
(i) A data processor and/or microprocessor (102).
(ii) An on-off switch (103).
(iii) A gyroscope device (104) (Gyroscope) for detecting and communicating pitch, or roll, or yaw of the Sensor (101);
(iv) Circuitry for external data communication with the Controller device (301) and in certain embodiments other Sensors (101), including a transmitter (105), receiver (106), and an antenna (107) that transforms electromagnetic energy to electrical signals provided to the receiver (106), and transforms electrical signals from the transmitter (105) to electromagnetic energy for transmission to remote radio receivers in the Controller (201) and/or other Sensors (101). The receiver (106) responds to the electrical signals from the antenna (107) to produce detected data for the controller device (108). The receiver (106) may include circuits such as filters and demodulators. The transmitter (105) responds to formatted data from the controller device (108) to provide the electrical signals to drive the antenna (107). The transmitter (105) may include circuits such as modulators and filters. The antenna (107), the receiver (106) and the transmitter (105) together form a radio communication circuit for two-way radio or other wireless communication with remote radio devices such as the Controller (301) and other Sensors (101).
(v) A device controller (108) to control the operation of each Sensor (101), which may be implemented as a processor, microprocessor, digital signal processor (DSP), or any other logic circuit or combination of circuits providing control functions; and may operate in response to data and to program instructions stored in the memory (109); and may also control the radio or other wireless communication circuit (105, 106, 107) by directing the tuning, activation, and deactivation of the circuit.
(vi) A memory unit or device (109) capable of storing data.
(vii) A vibration device (110) that causes the Sensor (101) to vibrate.
(viii) A speaker or other sound system (111) capable of emitting a variety of sounds (e.g., siren, beep, whistle, gong, etc.).
(ix) A battery of other power source and conductors (112) to operate the Sensor (101).
(x) Attachment systems or devices (113) by which the Sensor (101) may be affixed to or worn on or about a person's body. Such systems or devices may include adhesives, Velcro or similar backing, or clips.

Also as shown in FIG. 2, the Controller (301) may be a specially-made transceiver device, or a conventional device on which the biofeedback software program (401) may be installed (e.g., smart-phone, PDA or computer), in either case that would include a housing, circuitry and other components that may include all or some of the following:
(i) An internal display screen or external monitor (302) enabling a user to view menu options, obtain information about and program the Sensors (101).
(ii) User Interface (303) comprised of a keyboard, keypad, touchscreen, etc. to let user enter data and perform programming functions.
(iii) A memory unit or device (304) capable of storing data.
(iv) A speaker or other sound system (305) capable of emitting a variety of sounds (e.g., siren, beep, whistle, gong, etc.).
(v) A vibration device (306) that causes the Controller to vibrate.
(vi) A wireless communication system (307) (e.g., a transmitter, receiver and antenna) for Bluetooth, radio wave or other communications with each Sensor (101).
(vii) A data processor and/or microprocessor (308).
(viii) A device controller (309) that may be implemented as a processor, microprocessor, digital signal processor (DSP), or any other logic circuit or combination of circuits providing control functions. It may operate in response to data and to program instructions stored in the memory (304), and may control the radio or other wireless communication circuit (307) by directing the tuning, activation, and deactivation of the circuit.
(ix) A battery or other electric power source and conductors (310).
(x) An on-off switch (311).
(xi) Programs and operating systems (312) to enable the biofeedback system software and application (401) to be installed and run on the Controller (301), which software and application may be configured as computer readable program code and stored in the device's memory (304).

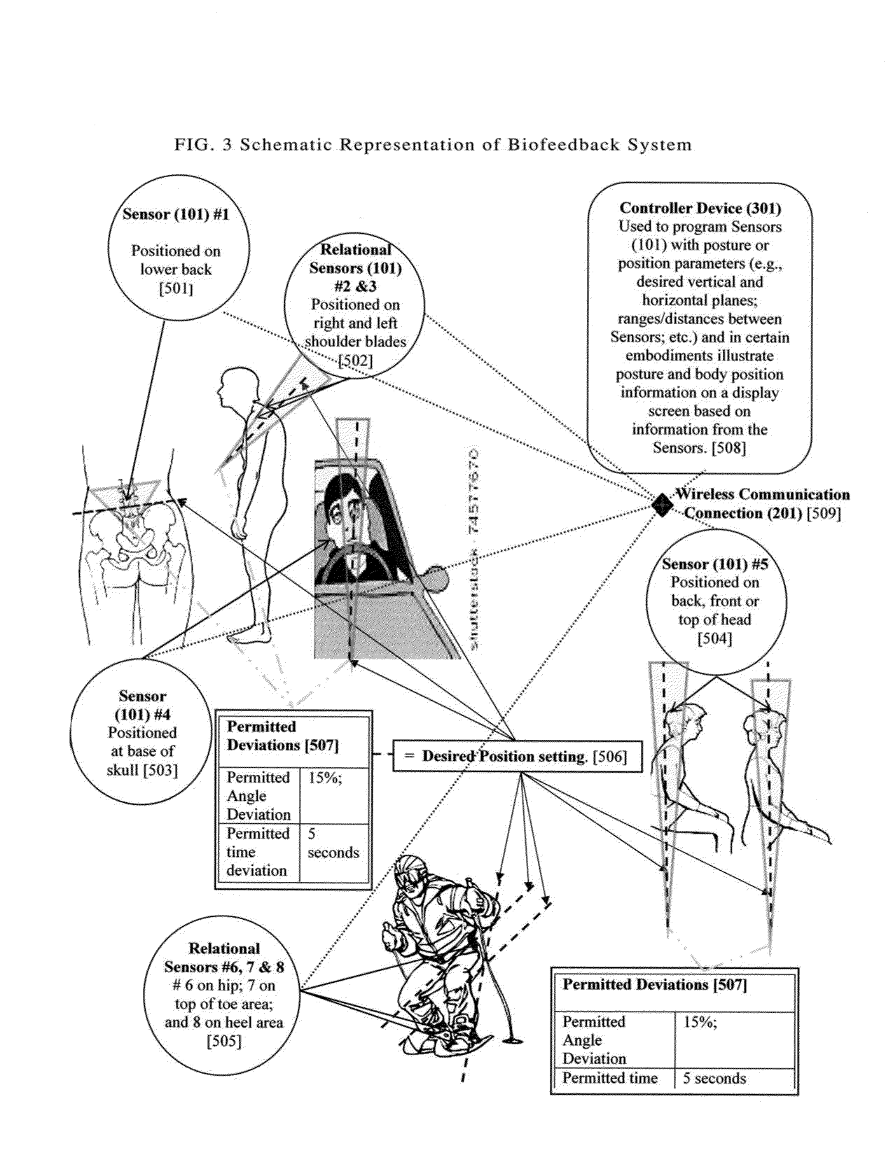

FIG. 3 illustrates a first embodiment of the present, novel biofeedback system (1, 5) for training users to attain and maintain targeted posture and/or other positions for different parts of the body. The training is provided through signals (i.e., vibration, sound and/or visual alarms and alerts) transmitted to a user and activated by measured changes in posture and position. Those signals are provided by one or more ‘Sensor’/transceiver biofeedback devices (101) and in some embodiments a ‘Controller’/transceiver biofeedback device (301). Each Sensor (101) is directly or indirectly (i.e., through clothes or accessories) attached to or worn on a specific part of a user's body (FIGS. 501-505). Those parts of the body are selected based on certain activities the user engages in where biofeedback information and/or training can enhance appearance, performance, health or safety. Selected activities may be as simple as standing or sitting with head held high and back erect (501 and 504); or as complicated as skiing with legs shoulder-width apart, knees bent at a 50 degree angle, and hips centered over the ball of the feet (505); or as critical as keeping a head up and staying awake while driving (503).

The Sensor detects and activates an alarm when a user attains or deviates from a targeted posture or position by gyroscopically measuring changes in the user's horizontal and/or vertical fields, and/or by wirelessly measuring distances between two or more of such devices. For example, if one Sensor is attached to a user's right shoulder, and a second is attached to a user's left shoulder, the Controller sets a target position when both shoulders are arched backward. The coordinates for that target position are represented by either or both (x) the distance between the devices and/or (y) the horizontal/vertical planes of each device. If the user slouches forward, the targeted distance is exceeded and planes change; either of which activates an alarm.

Biofeedback information and training is provided via the alarms from the Sensors (101) and in certain embodiments from the Controller (301). Those alarms are activated when either (x) the user deviates from a pre-set position or posture (506) (which can be adjusted with tolerances so that a deviation must be more than a pre-set degree and/or for more than a pre-set period of time (507) before the alarm is activated); or (y) the user achieves a pre-set, targeted position or posture (506). The devices are programmed so that the alarm will continue until a deviation is corrected. By way of example, if a user attaches a Sensor (101) to his head or neck while driving (503), vibratory and audible alarms will be activated in that Sensor, and in certain embodiments the Controller, if having fallen asleep, the user's head tilts so as to deviate by more than 15 degrees in any direction for more than 5 seconds from the Sensor's pre-set vertical plane (506, 507). As another example, if a skier (505) has Sensors (101) on the front and back of a ski boot, and on each hip and knee, vibratory and audible alarms will be activated in each Sensor and the Controller once the user attains a pre-set stance with the right and left feet 2′ apart, the knees bent at a 50 degree angle, and the hips centered over the heels (506).

Each Sensor (101) has a unique digital signal that allows the Controller (301, 508), through the wireless communication link (201, 509), to program settings for that Sensor (e.g., sounds, body position targets (506), permitted deviations (507), etc.) and to distinguish each Sensor from others. The Controller (301, 508) has unique digital signals that allow it to communicate with each Sensor (101). The Controller (301, 508) maintains its communication links to each Sensor (101) via a Bluetooth, infrared, radio or like short-range communication system (201, 509).

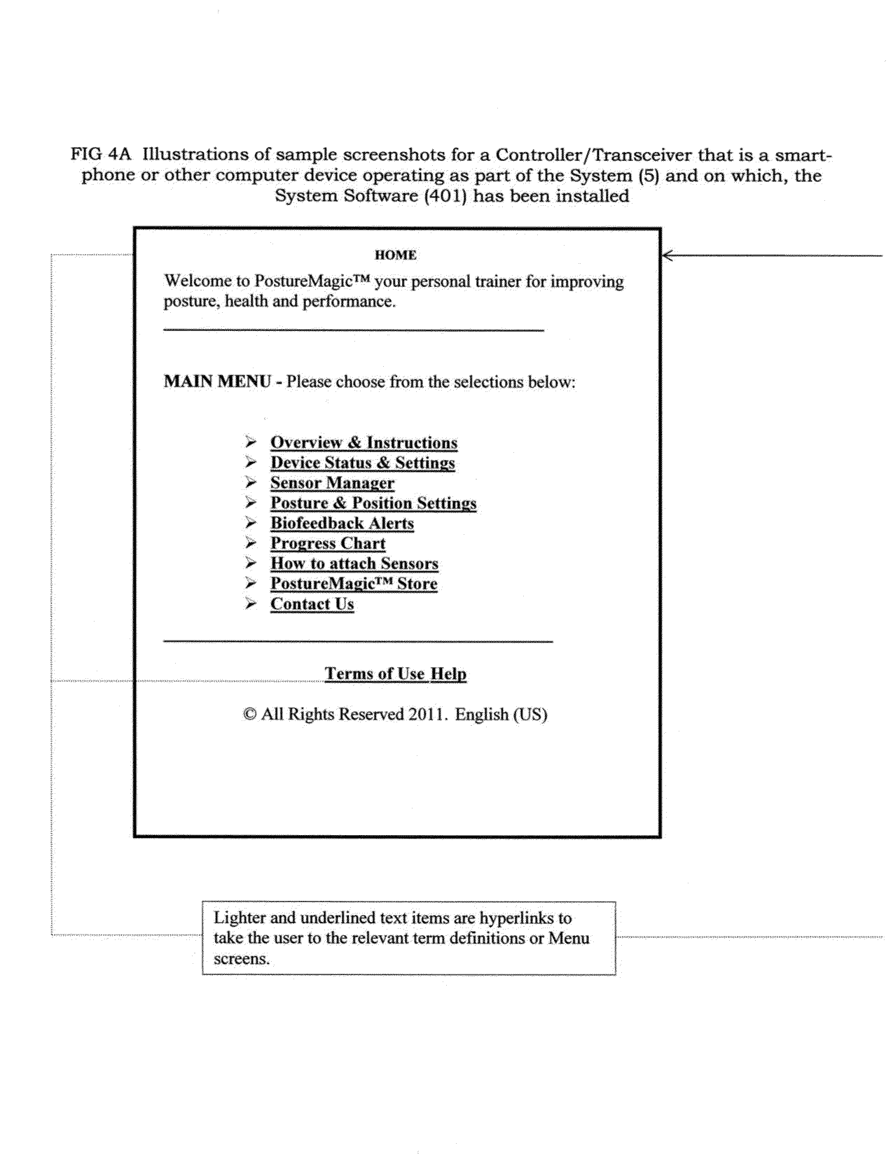

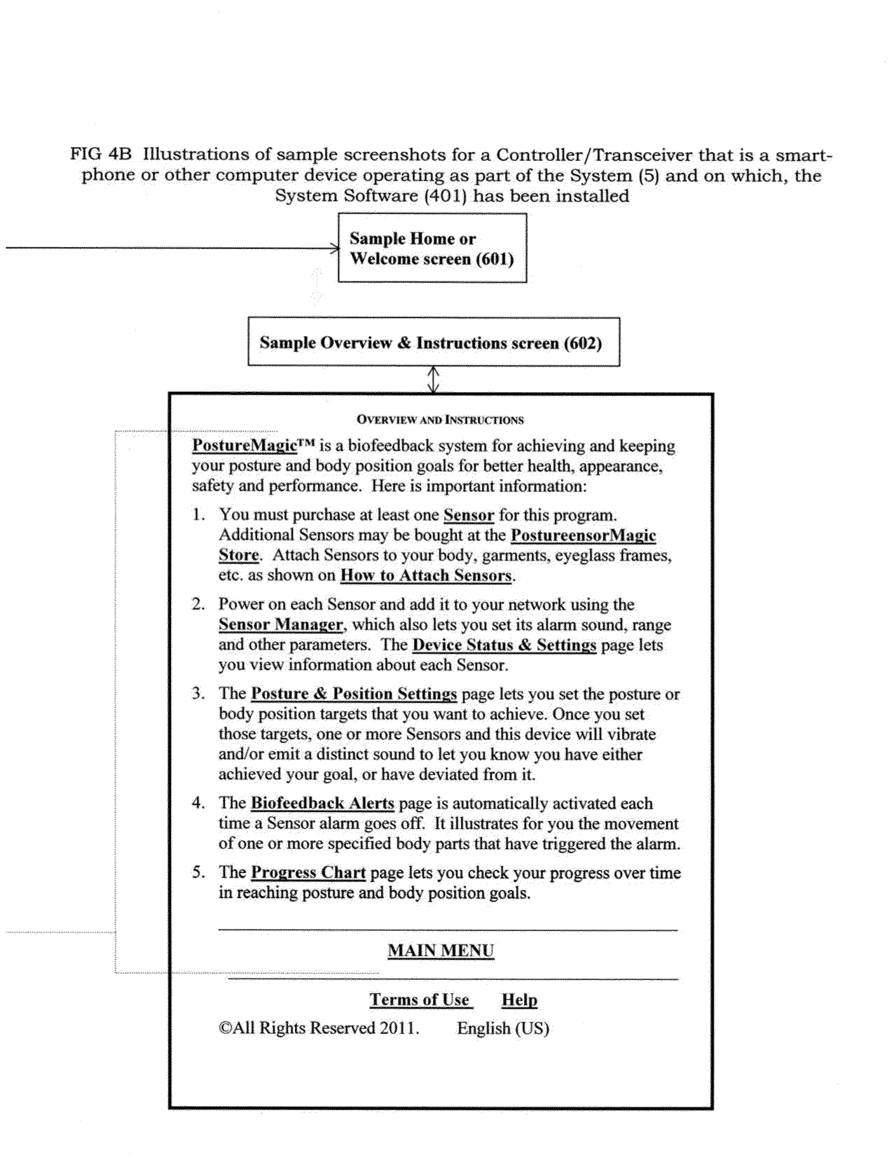

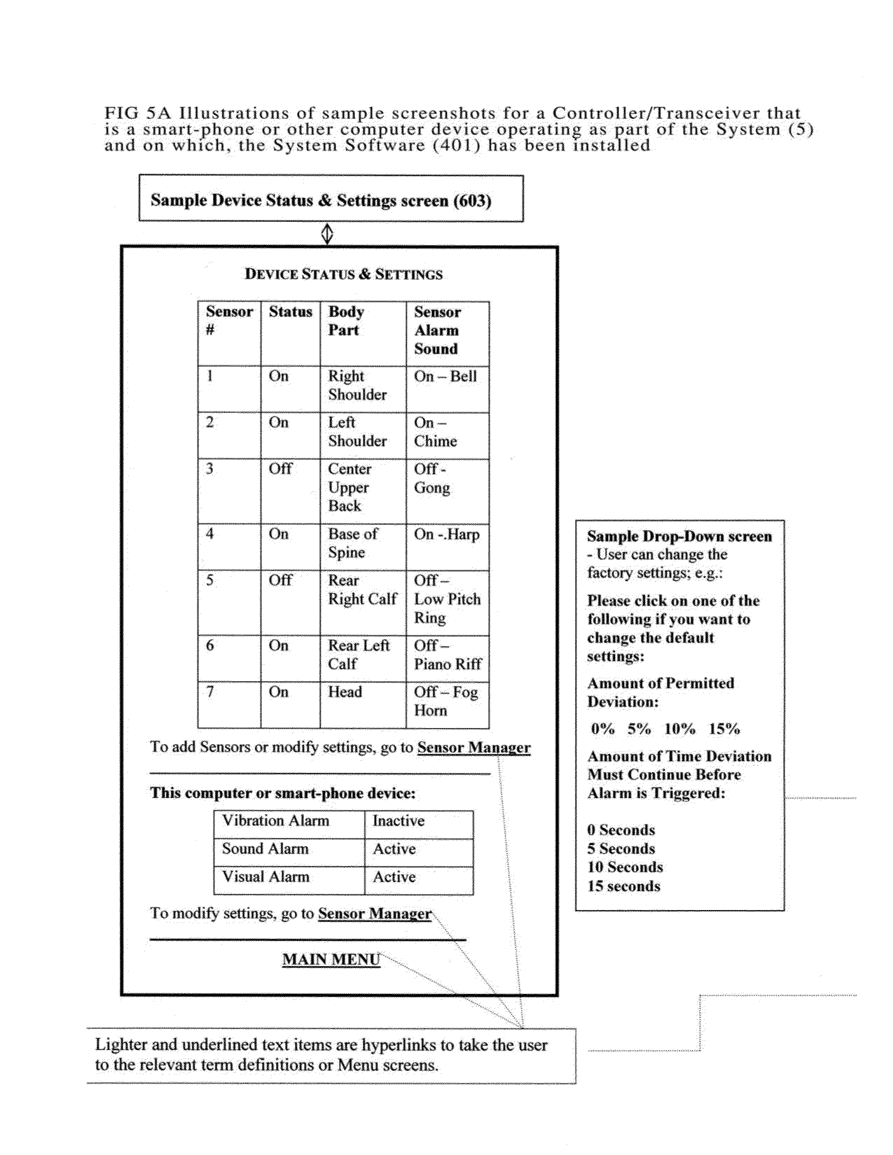

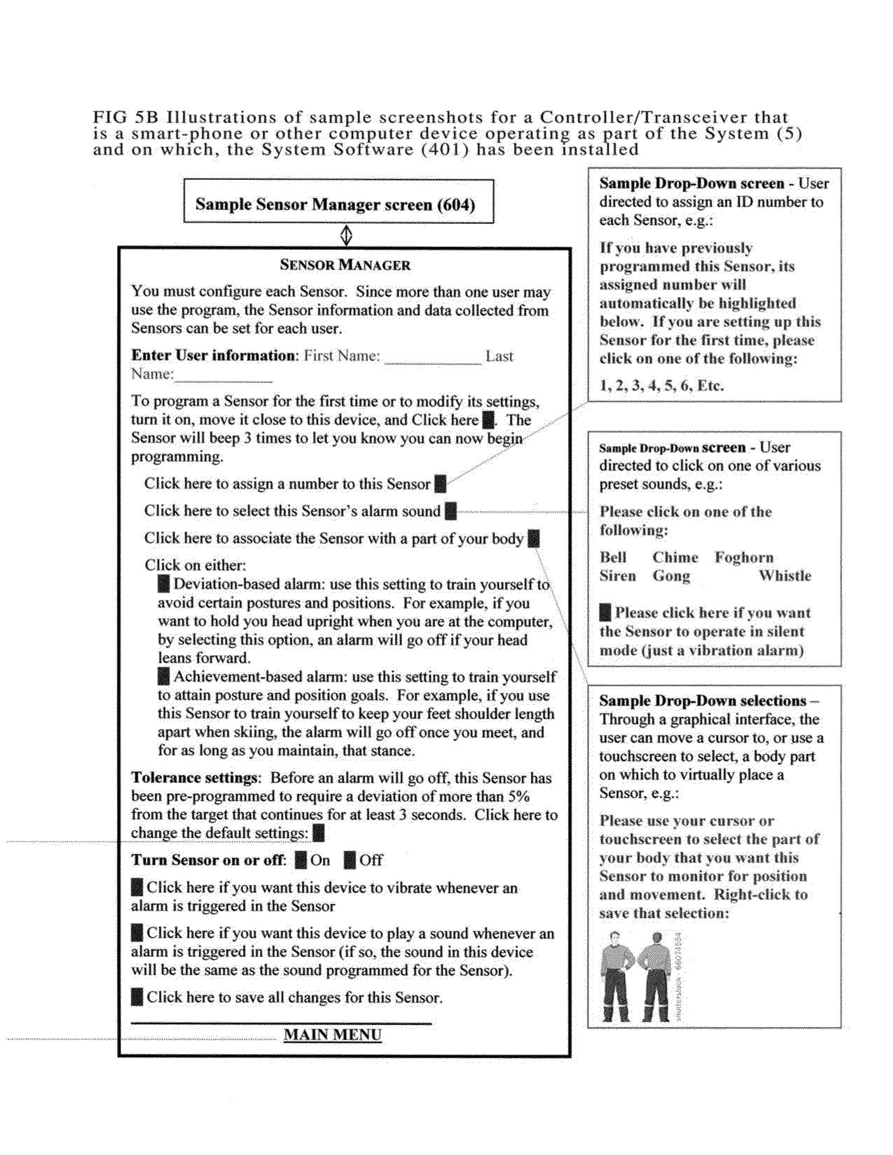

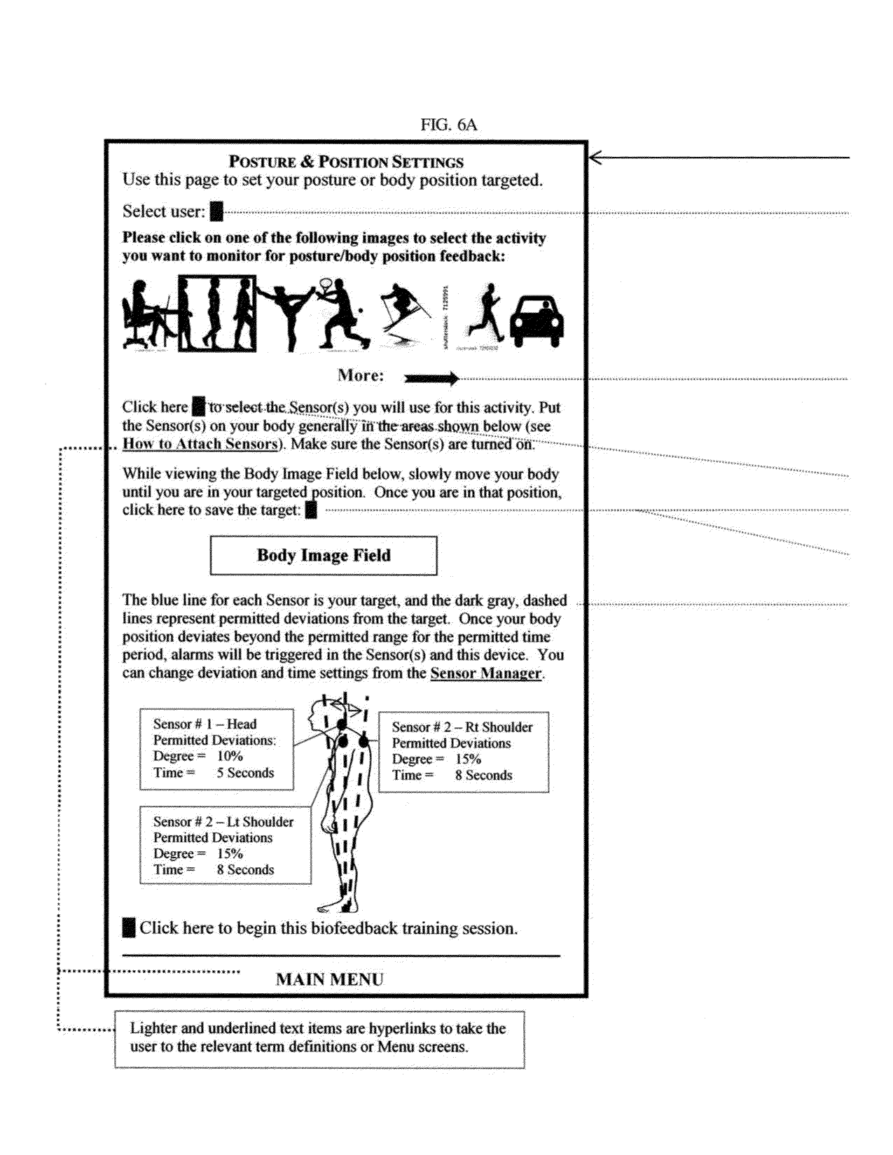

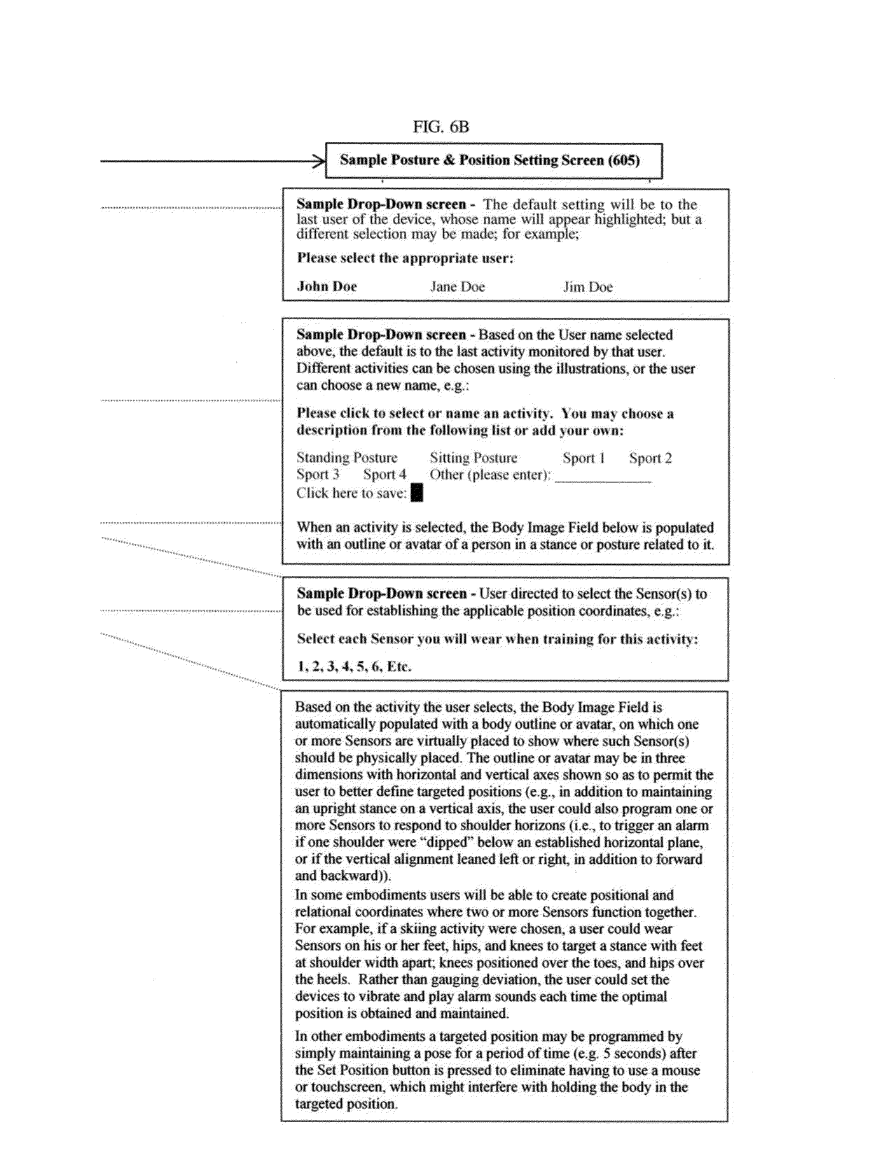

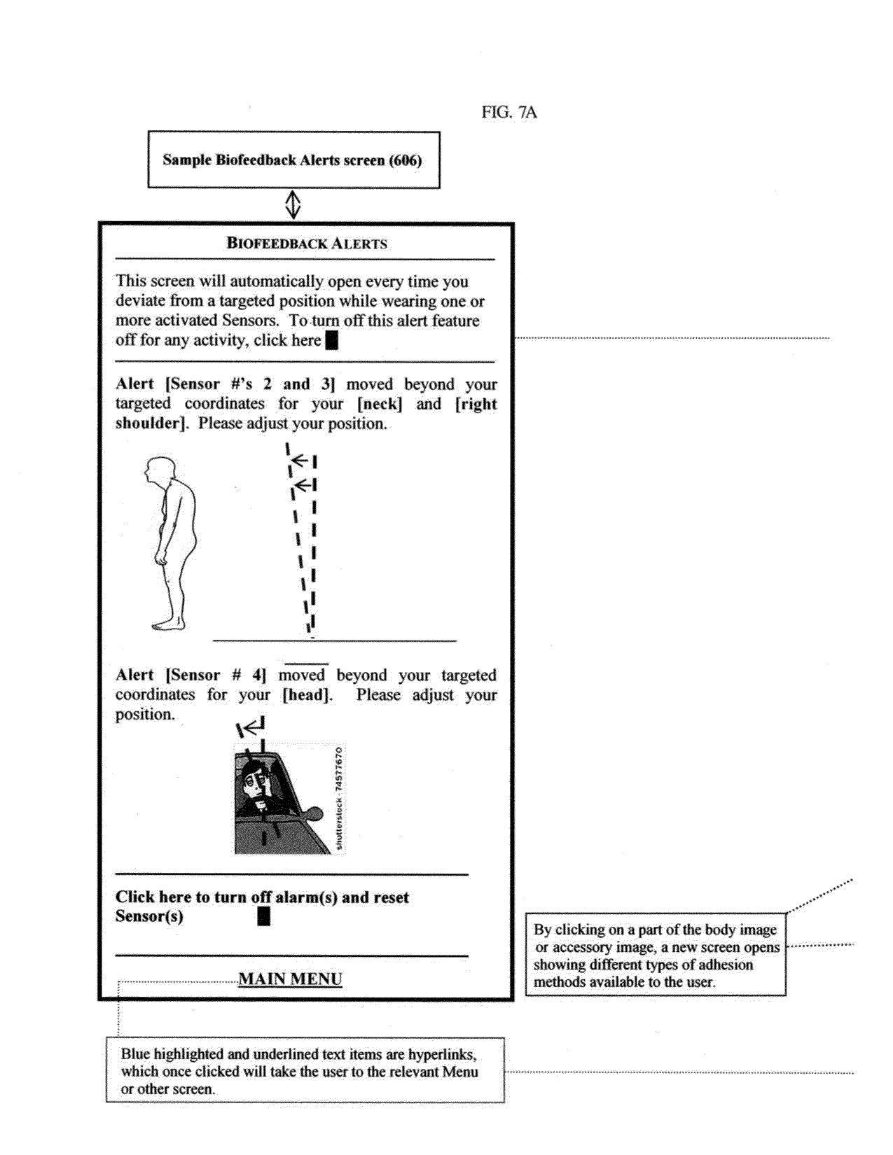

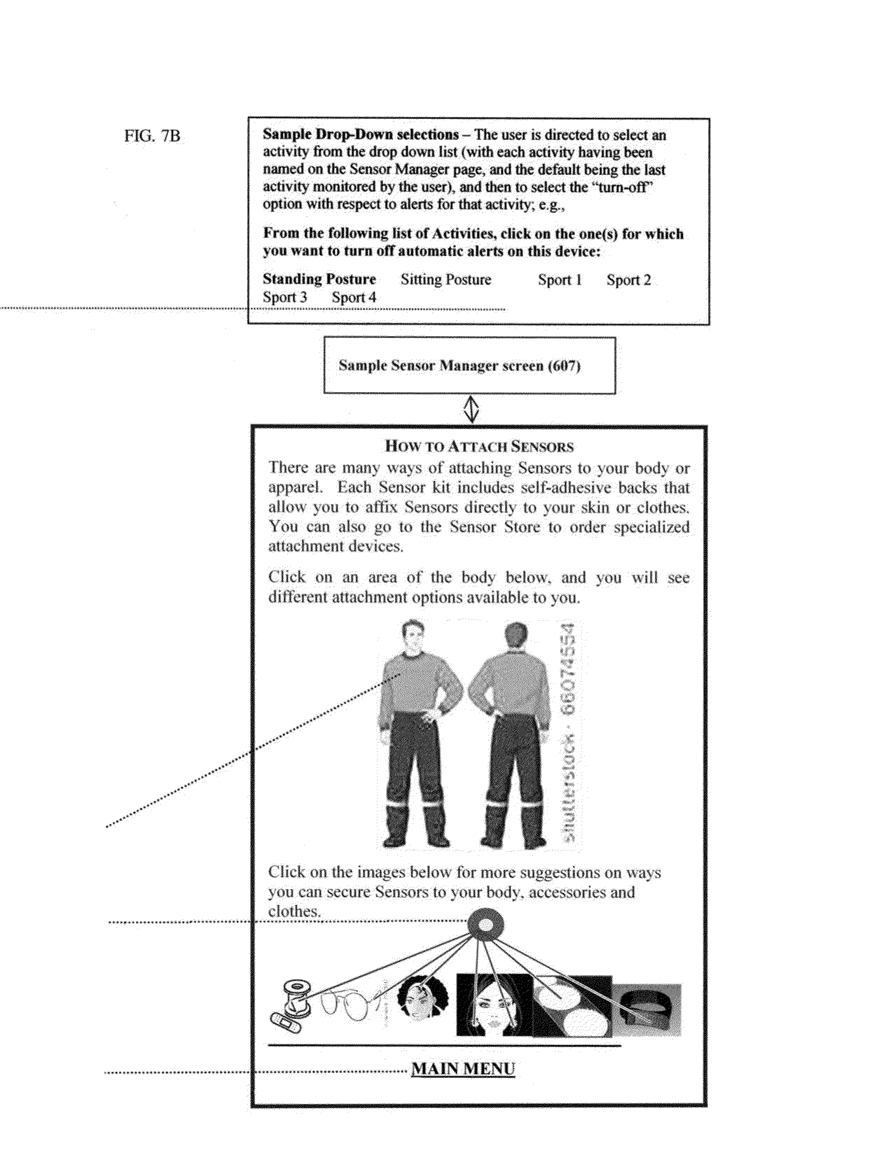

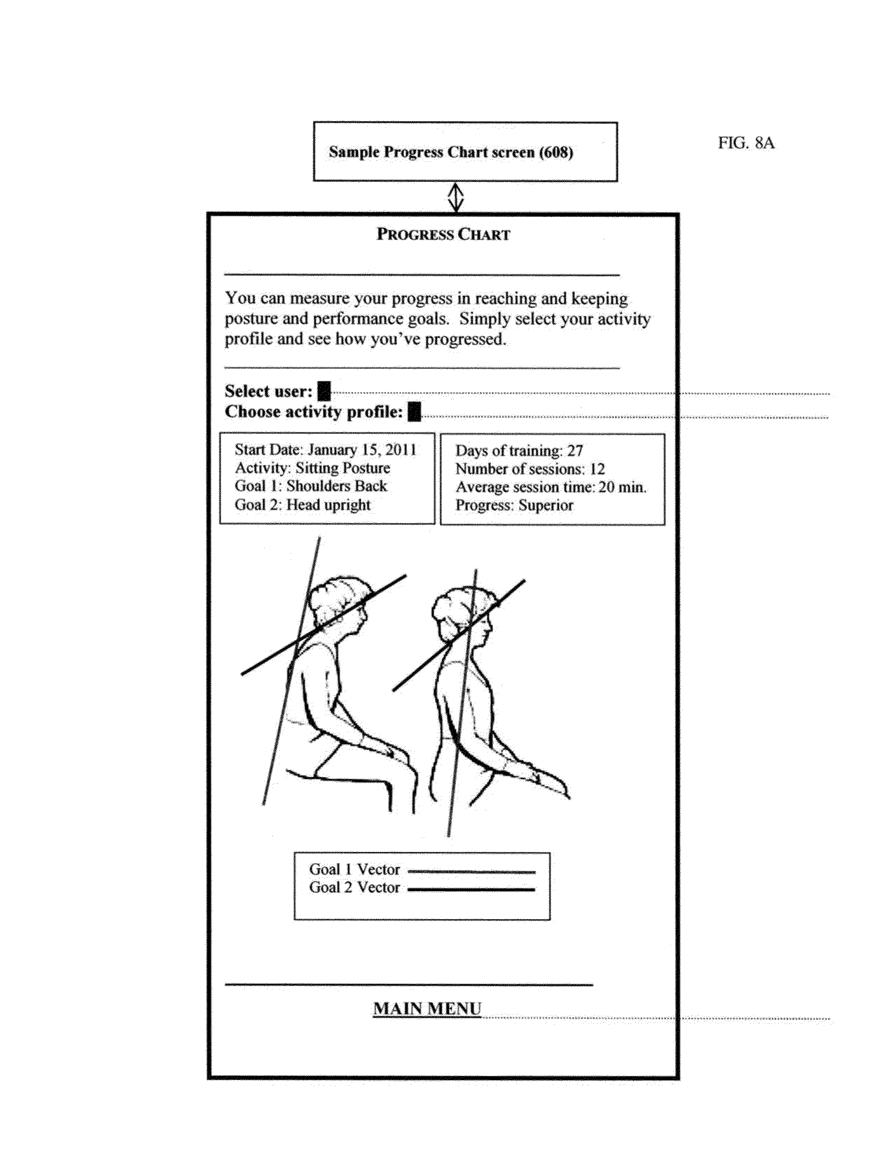

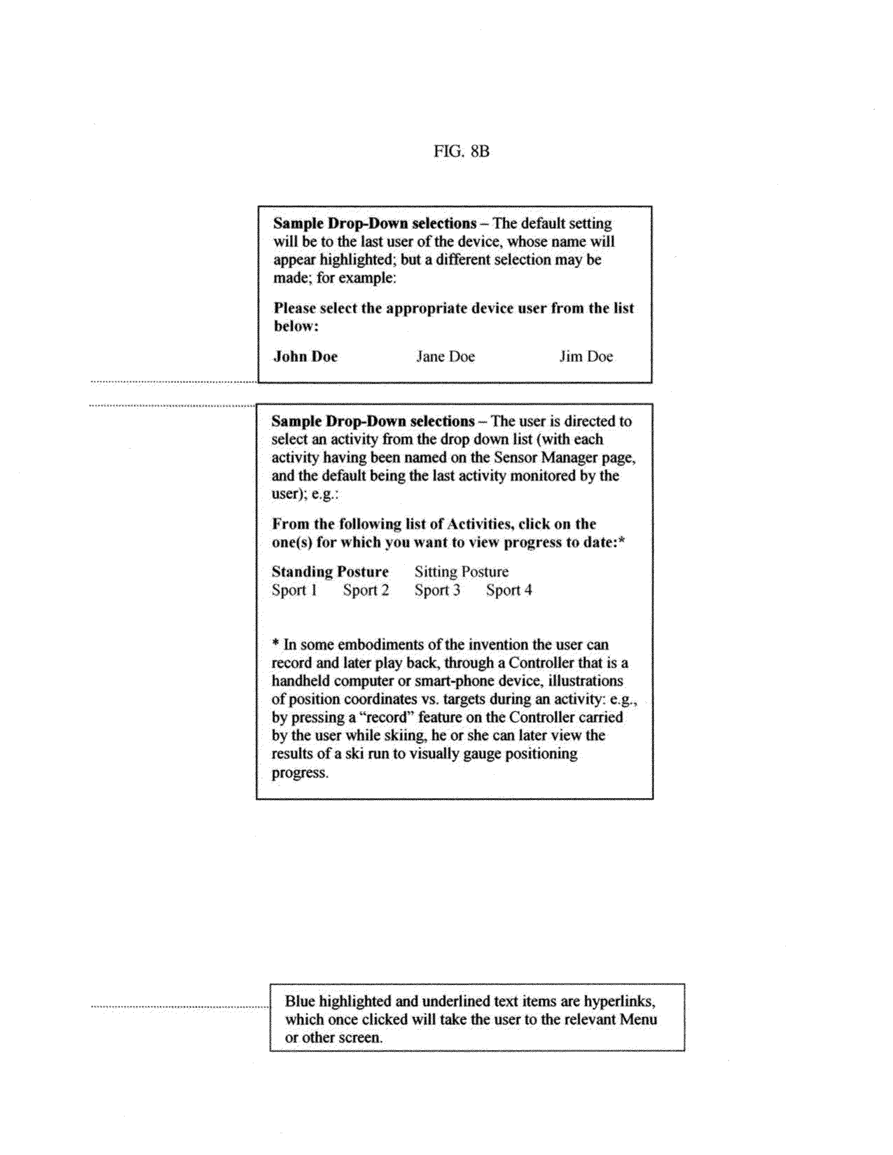

FIG. 4 illustrates one embodiment of a menu system of, and other screen shots for, the biofeedback system (5) that utilizes a device on which the biofeedback system software (401) has been installed (e.g., smart-phone, PDA, PC, laptop, etc.). FIG. 4 also illustrates certain functionalities of the system (5), and the “look and feel” of the biofeedback system software and application (401). It is to be noted that the illustrated menu system and screen shots are exemplary only. Other menu systems and screen shots/pages may be readily developed and provide additional functionalities and capabilities.

The Home Page/main menu (601) is a sample screen/page of the biofeedback system software program/application (401). The menu gives users various options for configuring and using the system and its devices (101 and 301). The selections of sub-pages accessed through the main menu (601) are illustrated in 602-608. By clicking on a link to a sub-page in the main menu (601), that sub-page appears on the screen. From any sub-page (602-608), by clicking on a Main Menu link, the user is returned to the Home page/Main Menu (601).

The System Overview and Instructions screen/page (602) provides the user with brief descriptions of different system (5) components and software features. This page instructs the user in the operation of the system (5) and describes its component devices and functions. By clicking on any definitional or descriptive item highlighted on this page, another sub-page opens on the screen with information or actions the user may perform.

The Device Status & Settings screen/page (603) provides the user with a detailed inventory of Sensors (101); the body parts/positions with which the Sensors are associated; and the sound/vibration settings for the Sensors and Controller (301). The page also allows the user to see the status (“on”/“off”) of each Sensor, and directs the user to modify Sensor (101) and Controller (301) settings and add information by clicking on the “Device Manager” link.

The Device Manager screen/page (604) provides various programming functions to manage system devices. Users can set parameters for the Sensors (101) and the Controller (301). Those parameters include (i) activating audible alarms in Sensors and the Controller, (ii) selecting a specific audible sound for each Sensor, (iii) activating a vibration alarm in the Controller, (iv) setting permitted deviations from targeted positions, (v) designating the period of time a deviation in position may be permitted to continue before an alarm is activated in a Sensor, (vi) assigning a number to each Sensor, and (vii) associating each Sensor with a specific part or area of the user's body.

The Posture & Position screen/page (605) allows users to program targeted positions and postures for Sensors (101). From this screen, a user can select a prescribed activity (e.g., standing, sitting, driving, skiing, etc.) which then opens a body avatar on which the user can virtually place one or more Sensors. With a Sensor attached to the user's body, he or she can then change positions in order to create a targeted posture or pose that can be saved. This page/screen also illustrates permitted deviations from targeted positions. Once a targeted position has been saved, the user can proceed to a biofeedback session, and will be “trained” by receiving vibration and/or sound and visual alarms/alerts whenever he or she deviates from, or achieves, that target.

The Biofeedback Alerts screen/page (606) provides additional biofeedback information to users. Unless de-activated by a user for an activity, the screen automatically opens each time an alarm is activated by one or more Sensors (101). The screen provides real-time notifications and visual displays of deviations from, or attainment of, targeted postures or positions.

The How to Attach Sensors screen/page (607) provides instructions for attaching Sensors (101) to parts of the user's body.

The Progress Chart screen/page (608) provides historical and real time data to a user about progress in reaching and maintaining targeted posture or position goals. From this screen/page, a user can select the activity he or she wishes to monitor, and can view a graphic representation of progress in relation to the established target for that activity. In certain embodiments, a user may record a real-time representation of posture/position changes during an activity, and play the recording back to gauge progress.

While the novel technology has been illustrated and described in detail in the drawings and foregoing description, the same is to be considered as illustrative and not restrictive in character. It is understood that the embodiments have been shown and described in the foregoing specifications in satisfaction of the best mode and enablement requirements. It is further understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the novel technology are desired to be protected.

While a particular embodiment of the present invention has been shown and described, modifications may be made. It is therefore intended in the appended claims to cover such changes and modifications which follow in the true spirit and scope of the invention.

What is claimed is:

1. A wireless, programmable biofeedback system, comprising:
one or more sensor/transceiver biofeedback devices;
a controller/transceiver biofeedback device; and
a wireless communication link that operationally connects the sensor/transceiver biofeedback device to the controller/transceiver biofeedback device, and in certain embodiments one or more other sensor/transceiver biofeedback devices;
a first antenna, receiver, transceiver and other circuitry operationally connected to the sensor/transceiver biofeedback device forming part of its wireless communications system;
a first processor, data processor and/or microprocessor operationally connected to the sensor/transceiver biofeedback;
a gyroscopic mechanism operationally connected to the sensor/transceiver biofeedback device;
a distance measuring mechanism operationally connected to the sensor/transceiver biofeedback device;
a first memory operationally connected to the sensor/transceiver biofeedback device;
a first data processor and/or microprocessor operationally connected to the first memory;
a predetermined signal stored in the first memory;
a first device controller operationally connected to the sensor/transceiver biofeedback device;
a first indicating means mechanism operationally connected to the sensor/transceiver biofeedback device;
an attachment element or system operationally connected to the sensor/transceiver biofeedback device;
a first battery or other source of electrical power and conducting wires operationally connected to the sensor/transceiver biofeedback device; and
a first on-off switch operationally connected to the sensor/transceiver biofeedback device;
wherein the sensor/transceiver biofeedback devices attached to a user's body by an attachment element detects changes in position of the part of the user's body to which the device is attached, and operationally communicate such changes wirelessly to the controller/transceiver biofeedback device;
wherein one or more indicating means provide stimulus to train and condition users to maintain or achieve certain postures and/or other body positions that the user can set, monitor and modify.

2. A biofeedback system software program comprising
application and database programs;
a graphical user interface;
computer readable codes;
source codes; and
user materials and documentation;
wherein a user of the biofeedback system operates, programs, monitors and stores information from, and enhances the functionality of the sensor/receiver biofeedback device and controller/transceiver biofeedback device.
